(19) United States
(12) Patent Application Publication
(10) Pub. No.: US 2018/0249991 A1
(43) Pub. Date: Sep. 6, 2018

(54) SHEATH ASSEMBLY FOR A TRANSURETHRAL PROCEDURE

(71) Applicant: NeoTract, Inc., Pleasanton, CA (US)

(72) Inventor: Jacqueline Nerney Welch, Moraga, CA (US)

(21) Appl. No.: 15/910,065

(22) Filed: Mar. 2, 2018

Related U.S. Application Data
(60) Provisional application No. 62/466,928, filed on Mar. 3, 2017.

(51) Int. Cl. A61B 17/00; A61B 1/015; A61B 1/12; A61B 1/307; A61B 1/00; A61M 31/00

(52) U.S. Cl. CPC A61B 17/00234; A61B 1/015; A61B 1/126; A61B 1/307; A61B 1/00135; A61B 2017/00274; A61B 2017/00296; A61B 2017/00336; A61B 2217/007; A61M 2205/075; A61M 31/00

(57) ABSTRACT

A sheath assembly for use in interventional procedures that creates a path for visualization. In one aspect, the sheath assembly includes a positive pressure device that can be manipulated to create fluid pressure and flow through an elongate tube portion of the sheath assembly.

### CROSS-REFERENCE TO RELATED APPLICATIONS

This application claims priority to and the benefit of U.S. Provisional Application No. 62/466,928 filed Mar. 3, 2017 entitled “Sheath Assembly For A Transurethral Procedure,” which is incorporated herein by reference in its entirety.

### BACKGROUND OF THE DISCLOSURE

The present disclosure relates generally to medical devices and methods, and more particularly to a sheath assembly for systems and associated methods for manipulating or retracting tissues and anatomical or other structures within the body of human or animal subjects for the purpose of treating diseases or disorders.

One example of a condition where it is desirable to lift, compress, manipulate, or remove a pathologically enlarged tissue is Benign Prostatic Hyperplasia (BPH). BPH is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule around the prostate gland may prevent the prostate gland from enlarging further. This causes the inner portions of the prostate gland to squeeze the section of the urethra that passes through the prostate gland, known as the prostatic urethra. This pressure on the prostatic urethra increases resistance to urine flow through the prostatic urethra. Thus, the urinary bladder has to exert more pressure to force urine through the increased resistance within the prostatic urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate, etc.

In addition to patients with BPH, LUTS may also be present in patients with prostate cancer, prostate infections, and chronic use of certain medications (e.g. ephedrine, pseudoephedrine, phenylpropanolamine, antihistamines such as diphenhydramine, chlorpheniramine, etc.) that cause urinary retention especially in men with prostate enlargement. Although BPH is rarely life threatening, it can lead to numerous clinical conditions including urinary retention, renal insufficiency, recurrent urinary tract infection, incontinence, hematuria, and bladder stones.

In developed countries, a large percentage of the patient population undergoes treatment for BPH symptoms. It has been estimated that by the age of 80 years, approximately 25% of the male population of the United States will have undergone some form of BPH treatment. At present, the available treatment options for BPH include watchful waiting, medications (phytotherapy and prescription medications), surgery and minimally invasive procedures.

For patients who choose the watchful waiting option, no immediate treatment is provided to the patient, but the patient undergoes regular exams to monitor progression of the disease. This is usually done on patients that have minimal symptoms that are not especially bothersome.

Surgical procedures for treating BPH symptoms include transurethal resection of prostate (TURP), transurethral electrovaporization of prostate (TVP), transurethral incision of the prostate (TUIP), laser prostatectomy and open prostatectomy. Minimally invasive procedures for treating BPH symptoms include transurethral microwave thermotherapy (TUMT), transurethral needle ablation (TUNA), interstitial laser coagulation (ILC), and prostatic stents.

The most effective current methods of treating BPH carry a high risk of adverse effects. These methods and devices either require general or spinal anesthesia or have potential adverse effects that dictate that the procedures be performed in a surgical operating room, followed by a hospital stay for the patient. The methods of treating BPH that carry lower risks of adverse effects are also associated with a lower reduction in the symptom score. While several of these procedures can be conducted with local analgesia in an office setting, the patient does not experience immediate relief and in fact often experiences worse symptoms for weeks after the procedure until the body begins to heal. Additionally all device approaches require a urethral catheter placed in the bladder, in some cases for weeks. In some cases catheterization is indicated because the therapy actually causes obstruction during a period of time post operatively, and in other cases it is indicated because of post-operative bleeding and potentially occlusive clot formation. While drug therapies are easy to administer, the results are suboptimal, take significant time to take effect, and often entail undesired side effects.

There have been advances in developing minimally invasive devices and methods for manipulate, modifying, and/or repositioning of tissues. However, further advances are necessary to ensure better visibility at an interventional site.

There remains a need for the development of new devices and methods that can be used to apply changes in fluid pressure or cause motion, dispersion, or removal of fluid, tissue, blood, gas, and bubbles in the tool, at an interventional site, or elsewhere in the body and to rinse a delivery device, other components, and/or the interventional site to provide improved visibility and/or operative conditions.

The present disclosure addresses these and other needs.

### SUMMARY

Briefly and in general terms, the present disclosure is directed towards a sheath assembly for use in transurethral or other medical procedures. In one approach, the sheath assembly is utilized with an apparatus and method for deploying an anchor assembly within a patient's body to accomplish interventional treatments. A delivery device is provided to access the anatomy targeted for the interventional procedure. The sheath assembly includes a structure that applies changes in fluid pressure and motion at an interventional site thereby providing visibility or enhancing operative conditions. In one embodiment, the sheath assembly is embodied in a system configured to treat BPH and rinses treatment components and devices.

In one particular aspect, the sheath assembly includes an elongate generally cylindrical tube portion extending from a main body or hub. The main body includes a pair of ports extending therefrom. Each port includes fluid management features, such as a stopcock and a stem portion sized and shaped to connect to one or more of a fluid source or a pressure source. An ergonomic positive pressure reservoir, such as a bulb or syringe, is further provided and configured to mate with the fluid management features of one or both ports. Manipulation or otherwise squeezing or compressing a positive pressure reservoir attached to a sheath port causes a positive pressure to be transmitted through the sheath assembly. Positive pressure exiting a terminal end of the tube portion provides fluid pressure and motion, for example, when the sheath assembly is place within or at an interventional site.

The sheath assembly can be employed as part of a system including a delivery device that accomplishes the delivery of a first or distal anchor assembly component at a first location within a patient's body and the delivery of a second or proximal anchor assembly component at a second location within the patient. The procedure can be viewed employing a scope inserted in the device. The scope can assume various configurations and can be employed with complementary structure assisting in the viewing function. The sheath assembly is configured to facilitate visualization by the scope at an interventional site. That is, the positive pressure device of the sheath assembly is manipulated such that positive pressure from the device displaces tissue, blood clots, and bubbles, at least partially clearing a path for visualization by the scope. The interventional device can be sized and shaped to be compatible inside a sheath up to 24 F, preferably a 19 F or 20 F sheath or smaller.

Various alternative methods of use are contemplated. The disclosed sheath apparatus and those of a similar nature used to access a natural body orifice can be used to improve visibility during an interventional procedure including improvement of flow of a body fluid through a body lumen, modifying the size or shape of a body lumen or cavity, administering contrast solution, administering medications, administering vasomanipulative drugs, administering drugs to accomplish other localized effects, creating transient fluid or pressure waves for therapeutic purpose, treating prostate enlargement, treating urinary incontinence, supporting or maintaining positioning of a tissue, closing a tissue wound, organ or graft, performing a cosmetic lifting or repositioning procedure, forming anastomotic connections, and/or treating various other disorders where a natural or pathologic tissue or organ is pressing on or interfering with an adjacent anatomical structure. Further, it is contemplated that the apparatus to gain access to the body might enter through a non-natural orifice such as a puncture site of a vessel or organ.

Other features and advantages of the present disclosure will become apparent from the following detailed description, taken in conjunction with the accompanying drawings, which illustrate, by way of example, the principles of the disclosure.

### BRIEF DESCRIPTION OF THE DRAWINGS

FIG. 1 is a side view, depicting one embodiment of a sheath assembly of the present disclosure.

FIG. 2 is a side view depicting one embodiment of an anchor delivery system.

FIG. 3 is a perspective view depicting the anchor delivery system of FIG. 2 inserted through the sheath assembly shown in FIG. 1 and with the assemblies inserted within a patient's body.

FIG. 4 shows a coronal section through the lower abdomen of a male human suffering from BPH showing a hypertrophied prostate gland.

FIG. 5 shows a coronal section through the lower abdomen of a male human suffering from BPH showing a hypertrophied prostate gland treated with an embodiment of the device of the present disclosure.

FIG. 6 shows a side view of an embodiment of the retainer shown in FIG. 5.

FIG. 7 shows the various steps of a method of treating a prostate gland by the retainer shown in FIG. 6.

FIG. 8 shows the various steps of a method of treating a prostate gland by the retainer shown in FIG. 6.

FIG. 9 shows the various steps of a method of treating a prostate gland by the retainer shown in FIG. 6.

FIG. 10 shows the various steps of a method of treating a prostate gland by the retainer shown in FIG. 6.

FIG. 11 shows the various steps of a method of treating a prostate gland by the retainer shown in FIG. 6.

FIG. 12 shows the various steps of a method of treating a prostate gland by the retainer shown in FIG. 6.

FIG. 13 shows the various steps of a method of treating a prostate gland by the retainer shown in FIG. 6.

FIG. 14 shows one embodiment of a positive pressure reservoir.

### DETAILED DESCRIPTION OF THE PREFERRED EMBODIMENTS

Turning now to the figures, which are provided by way of example and not limitation, the present disclosure is directed to sheath assembly for use in interventional procedures. In one particular aspect, the sheath assembly is used as part of a system including a device configured to deliver multiple anchor assemblies within a patient's body for treatment purposes. The disclosed system can be employed for various medical purposes including, but not limited to retracting, lifting, compressing, approximating, supporting, remodeling, manipulating, removing, or repositioning tissues, organs, anatomical structures, grafts, or other material within a patient's body. Such tissue manipulation is intended to facilitate the treatment of diseases or disorders such as the displacement, compression and/or retraction of the body tissue.

In an aspect of the present disclosure, the sheath assembly includes a main body or hub supporting an elongate member. The elongate member is characterized by a comparatively low profile that is suited to navigate body anatomy to reach an interventional site. That is, the elongate member is capable of being used in minimally invasive medical procedures. The sheath assembly can be employed as part of a system including an endoscope to provide the ability to view the interventional procedure. In this context, the sheath assembly operates to clear a path for visualization through the endoscope.

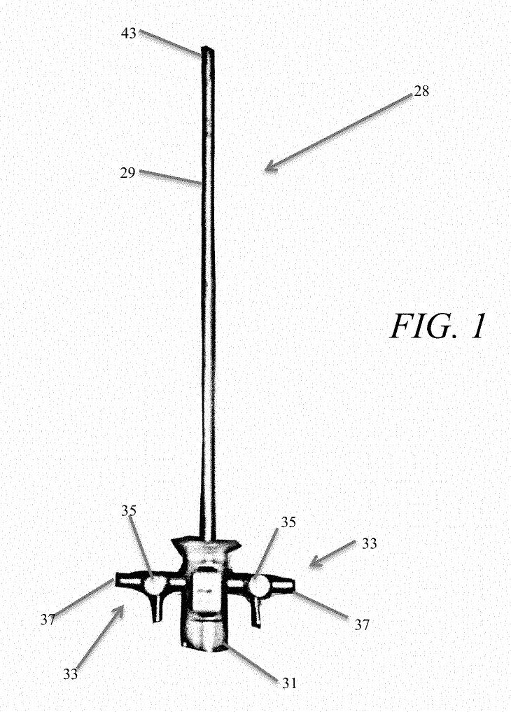

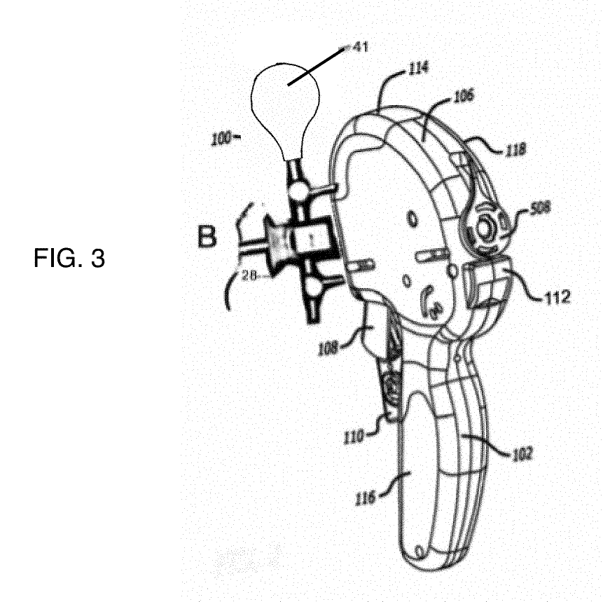

Referring now to FIG. 1, the sheath assembly 28 includes an elongate portion 29 extending from a main body or hub 31. In some embodiments, the elongate portion 29 is a generally cylindrical tube. The hub 31 includes a pair of ports 33 extending therefrom. Each port 33 includes a fluid management device, such as a stopcock 35 and a stem portion 37, sized and shaped to connect to one or more of a fluid source or a pressure source. A positive pressure reservoir, such as bulb 41, is further provided and configured to mate with the stem portion 37 of one or both ports 33 (See FIG. 3). Manipulation or otherwise squeezing or compressing a positive pressure bulb 41 attached to a sheath port 33 causes a positive pressure to be transmitted through the sheath assembly 28. Positive pressure at and/or exiting a terminal end 43 of the tube portion 29 provides fluid pressure and motion, for example, when the sheath assembly is place within or at an interventional site.

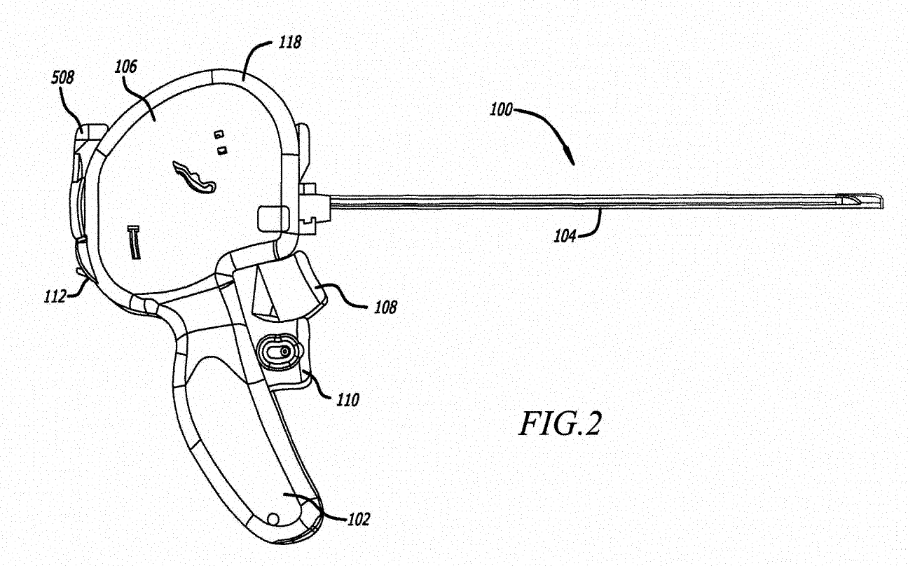

Referring now to FIGS. 2 and 3, there is shown one embodiment of a delivery device 100 that can be employed in combination with the sheath assembly 28. This device is configured to include structure that is capable of both gaining access to an interventional site as well as assembling and implanting one or more anchor assemblies or implants within a patient's body. The delivery device 100 can be configured to assemble and implant a single anchor assembly or implant a single bodied anchor or multiple anchors or anchor assemblies. The device is further contemplated to be compatible for use with a 19 F or 20 F sheath assembly 28. The device additionally includes structure configured to receive a conventional remote viewing device (e.g., an endoscope) so that the steps being performed at the interventional site can be observed. As shown in FIG. 3, the delivery device 100 is inserted within the sheath assembly 28 and placed within a patient's body B. In one embodiment, a positive pressure bulb 41 is attached to the sheath assembly and configured to provide positive pressure through the sheath assembly 28 as needed during an interventional procedure.

The anchor delivery device 100 includes a handle assembly 102 connected to elongate member 104. Elongate member 104 can house components employed to construct an anchor assembly and is sized to fit into a 19 F or 20 F sheath assembly 28 for patient tolerance during a procedure in which the patient is awake rather than under general anesthesia. The assembly is intended to include structure to maintain its positioning within anatomy.

The anchor delivery device 100 further includes a number of subassemblies. A handle case assembly 106 including mating handle parts that form part of the handle assembly 102. The handle assembly 102 is sized and shaped to fit comfortably within an operator's hand and can be formed from conventional materials. Windows can be formed in the handle case assembly 106 to provide access to internal mechanisms of the device so that a manual override is available to the operator in the event the interventional procedure needs to be abandoned.

In one embodiment, the delivery device 100 is equipped with various activatable members that facilitate assembly and delivery of an anchor assembly at an interventional site. A needle actuator 108 is provided and effectuates the advancement of a needle assembly to an interventional site. In one approach, the needle assembly moves through a curved trajectory and exits the needle housing in alignment with a handle element, and in particular embodiments, in alignment with the grip. In various other embodiments, the needle housing is oriented such that the needles exits the housing at either the two o'clock or ten o'clock positions relative to a handle grip that is vertical. A needle retraction lever assembly 110 is also provided and when actuated causes the needle assembly to be withdrawn and expose the anchor assembly.

In one particular, non-limiting use in treating a prostate, the elongate member 104 of a delivery device is, as stated, inserted within a sheath assembly 28 placed within a urethra (UT) leading to a urinary bladder (UB) of a patient. The patient is positioned in lithotomy. The elongate member 104 is advanced within the patient until a leading end thereof reaches a prostate gland (PG) and extends beyond the terminal end 43 of the sheath assembly 28. In a specific approach, the side(s) (or lobe(s)) of the prostate to be treated is chosen while the device extends through the bladder and the device is turned accordingly. The inside of the prostate gland, including the adenoma, is spongy and compressible and the outer surface, including the capsule, of the prostate gland is firm. By the physician viewing with an endoscope, he/she can depress the urethra into the prostate gland compressing the adenoma and creating the desired opening through the urethra. To accomplish this, the physician rotates the tool. The physician then pivots the tool laterally about the pubic symphysis PS relative to the patient's midline. The delivery device is at this stage configured in a ready state. The needle actuator 108 and the needle retracting lever 110 are in an inactivated position.

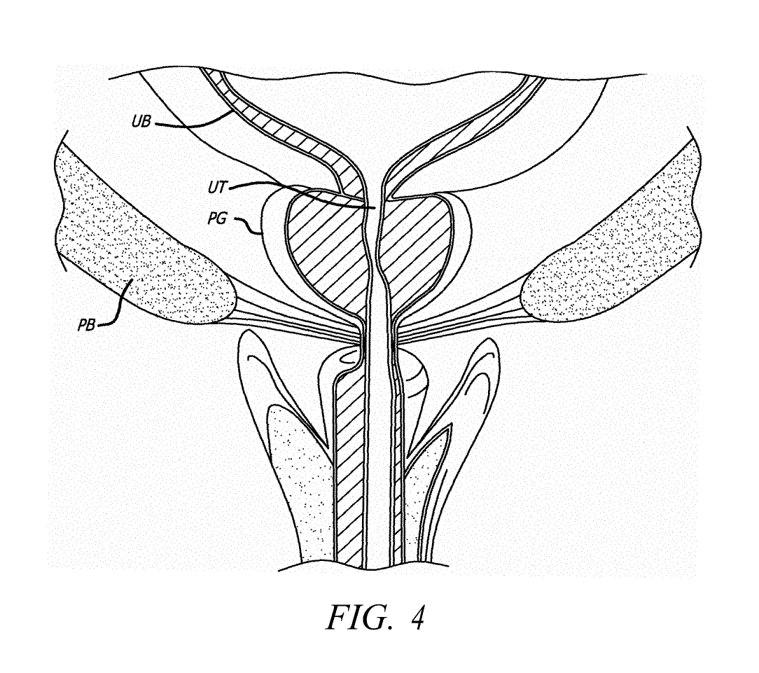

FIG. 4 shows a coronal section (i.e., a section cut approximately in the plane of the coronal suture or parallel to it) through the lower abdomen of a male human suffering from BPH showing a hypertrophied prostate gland. As depicted in FIG. 4, the urinary bladder UB is a hollow muscular organ that temporarily stores urine. It is situated behind the pubic bone PB. The lower region of the urinary bladder has a narrow muscular opening called the bladder neck, which opens into a soft, flexible, tubular organ called the urethra UT. The muscles around the bladder neck are called the internal urethral sphincter. The internal urethral sphincter is normally contracted to prevent urine leakage. The urinary bladder gradually fills with urine until full capacity is reached, at which point the sphincters relax. This causes the bladder neck to open, thereby releasing the urine stored in the urinary bladder into the urethra. The urethra conducts urine from the urinary bladder to the exterior of the body. The urethra begins at the bladder neck and terminates at the end of the penis. The prostate gland PG is located around the urethra at the union of the urethra and the urinary bladder. In FIG. 4, the prostate gland is hypertrophied (enlarged). This causes the prostate gland to press on a region of the urethra. This in turn creates an undesired obstruction to the flow of urine through the urethra.

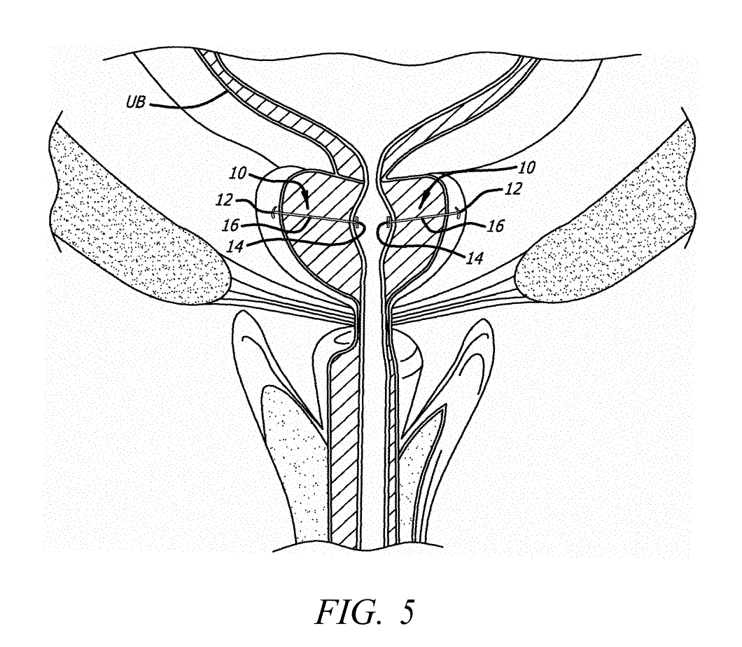

FIG. 5 shows a coronal section through the lower abdomen of a male human suffering from BPH showing a hypertrophied prostate gland treated with an embodiment of the device of the present disclosure. It has been discovered that the enlarged prostate gland is compressible and can be retracted so as to relieve the pressure from the urethra. In accordance with one embodiment of the present disclosure, a retaining device can be placed through the prostate gland in order to relieve the pressure on the urethra. In FIG. 5, a retainer 10 is implanted in the prostate gland. Retainer 10 comprises a distal anchor 12 and a proximal anchor 14. Distal anchor 12 and a proximal anchor 14 are connected by a connector 16. The radial distance from the urethra to distal anchor 12 is greater than the radial distance from the urethra to proximal anchor 14. The distance or tension between the anchors is sufficient to compress, displace or change the orientation of an anatomical region between distal anchor 12 and proximal anchor 14. The connector 16 can be inelastic so as to maintain a constant force or distance between the proximal and distal anchors or be elastic so as to attempt to draw the proximal and distal anchors closer together. In the embodiment shown in FIG. 5, distal anchor 12 is located on the outer surface of the capsule of prostate gland CP and acts as a capsular anchor. Alternatively, distal anchor 12 may be embedded inside the tissue of prostate gland PG or in the surrounding structures around the prostate such as periosteum of the pelvic bones, within the bones themselves, pelvic fascia, coopers ligament, muscles traversing the pelvis or bladder wall. Also, in the embodiment shown in FIG. 5, proximal anchor 14 is located on the inner wall of urethra UT and acts as a urethral anchor. Alternatively, proximal anchor 14 may be embedded inside the tissue of prostate gland PG or surrounding structures as outlined above. Distal anchor 12 and proximal anchor 14 are implanted in the anatomy such that a desired distance or tension is created in connector 16. This causes distal anchor 12 and proximal anchor 14 to retract or compress a region of prostate gland PG to relieve the obstruction shown in FIG. 4. In FIG. 5, two retainers 10 are implanted in prostate gland PG. Each retainer 10 is implanted in a lateral lobe (side lobe) of prostate gland PG. The various methods and devices disclosed herein may be used to treat a single lobe or multiple lobes of the prostate gland or other anatomical structures. Similarly, two or more devices disclosed herein may be used to treat a single anatomical structure. For example, a lateral lobe of prostate gland PG may be treated using two retainers 10. One or more retainers may be deployed at particular angles to the axis of the urethra to target one or more lateral lobes and/or middle lobe of the prostate gland. In one embodiment, retainer 10 is deployed between the 1 o'clock and 3 o'clock position relative to the axis of the urethra to target the left lateral lobe of the prostate gland. In another embodiment, retainer 10 is deployed between the 9 o'clock and 11 o'clock position relative to the axis of the urethra to target the right lateral lobe of the prostate gland. In another embodiment, retainer 10 is deployed between the 4 o'clock and 8 o'clock position relative to the axis of the urethra to target the middle lobe of the prostate gland.

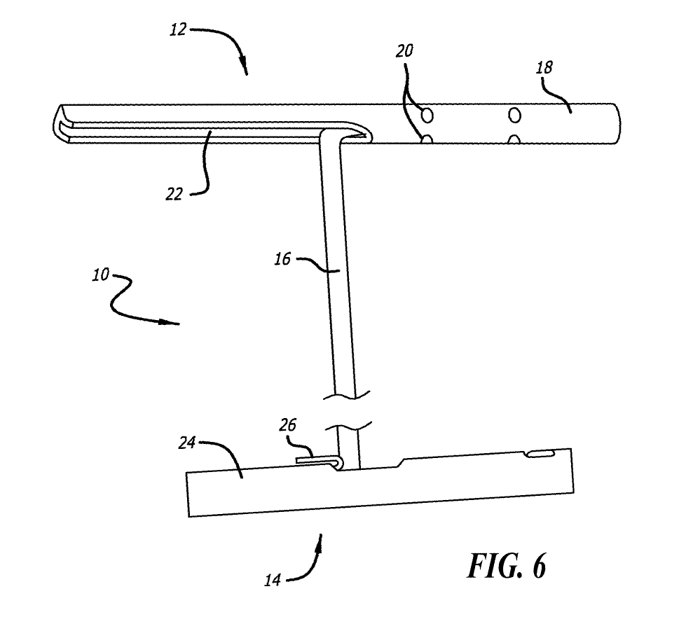

FIG. 6 shows a side view of one embodiment of the retainer shown in FIG. 5. FIG. 6 shows retainer 10 comprising distal anchor 12 and proximal anchor 14. Distal anchor 12 and proximal anchor 14 are connected by connector 16.

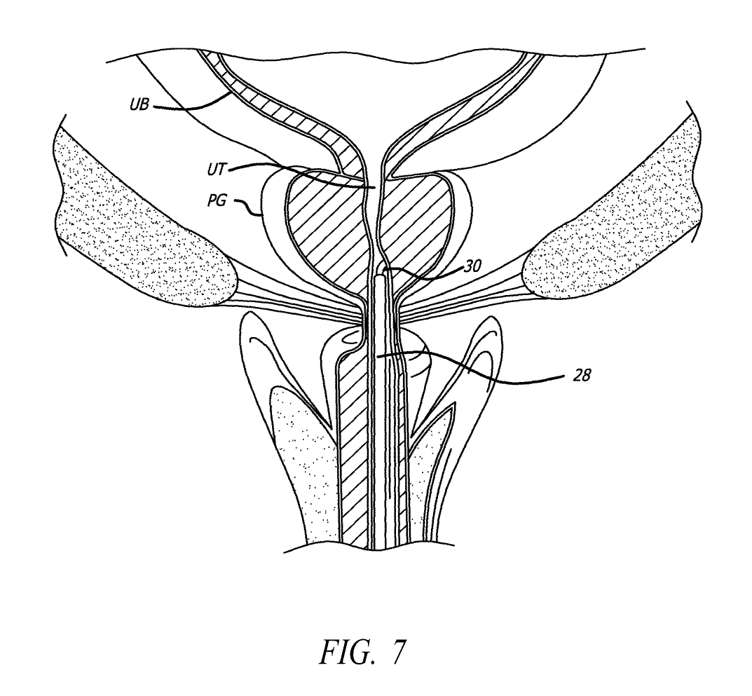

FIGS. 7 through 13 show the various steps of a method of treating a prostate gland by the retainer shown in FIG. 6. Similar methods may be also used to deploy retainer or compression devices in other anatomical structures. In the step shown in FIG. 7, the sheath 28 is introduced into the urethra (trans-urethrally). Sheath 28 is advanced through urethra UT such that the distal end of sheath 28 is positioned near a region of urethra UT that is obstructed by a hypertrophied prostate gland PG. Distal anchor delivery device 30 is introduced through sheath 28. Distal anchor delivery device 30 can be placed in the sheath 28 after the distal end of sheath 28 is positioned near the region of the urethra UT that is obstructed or the distal anchor delivery device 30 can be pre-loaded in the sheath 28 before positioning of the sheath 28. Distal anchor delivery device 30 is advanced through sheath 28 such that the distal end of distal anchor delivery device 30 emerges out of the distal end of sheath 28. Distal anchor delivery device 30 is oriented such that a working channel opening of distal anchor delivery device 30 points towards a lateral lobe of prostate gland PG. Notably, with the sheath 28 in place, the positive pressure bulb 41 can be at any time attached to the one of the stems 37 of a port and then manipulated to provide positive pressure and fluid motion to provide a path for visibility by a scope or other visualization device (See FIGS. 1 and 3). The positive pressure from the bulb 41 can be employed to displace tissue, blood, bubbles or other material or structure existing at the interventional site.

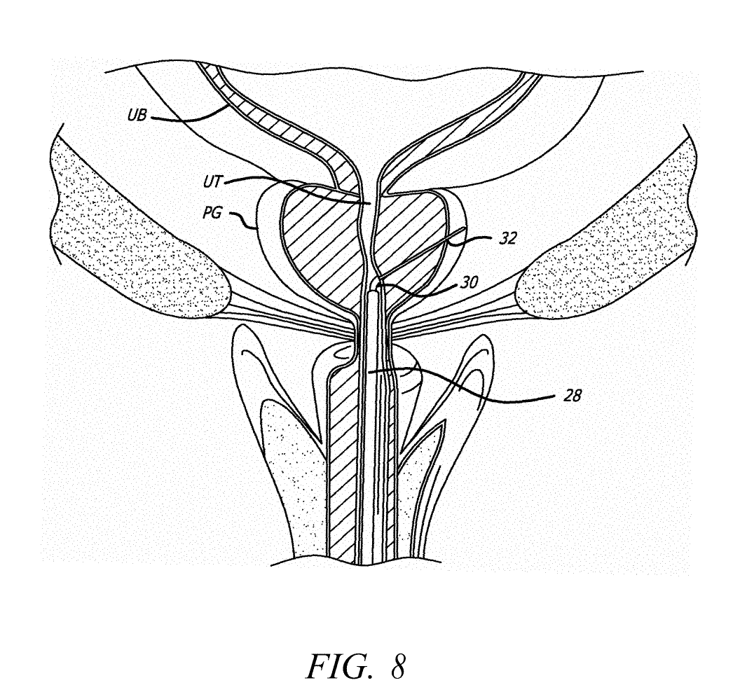

In the step shown in FIG. 8, a needle 32 is introduced through distal anchor delivery device 30. Needle 32 can be placed in distal anchor delivery device after the distal anchor delivery device 30 is advanced through sheath 28 or the needle 32 can be pre-loaded in the distal anchor delivery device 30. In one embodiment, needle 32 is a 20-gauge needle. Needle 32 is advanced through distal anchor delivery device 30 such that it emerges through the working channel opening. Needle 32 is further advanced such that it penetrates through the tissue of prostate gland PG and the distal end of needle 32 emerges out of the capsule of prostate gland CP.

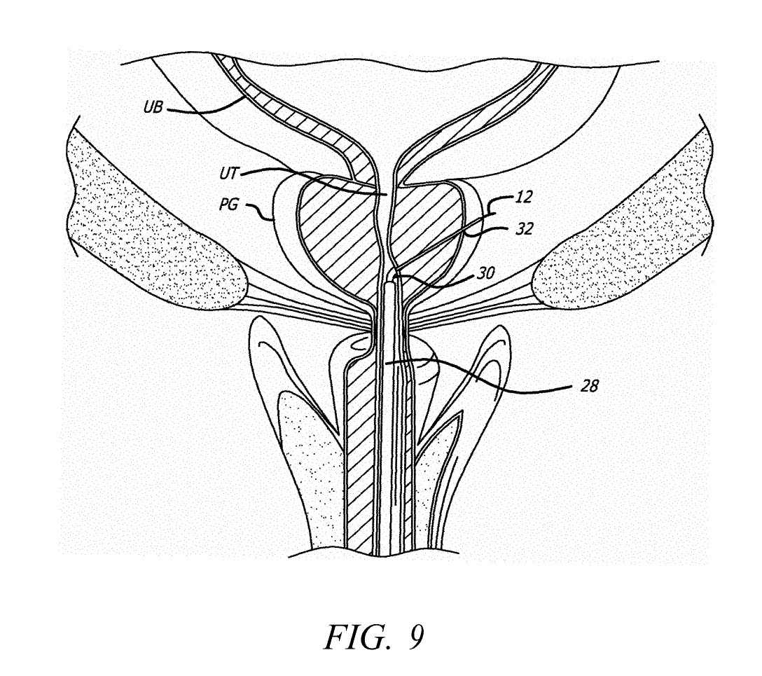

In the step shown in FIG. 9, distal anchor 12 connected to connector 16 is advanced through needle 32. Distal anchor 12 can be pre-loaded in needle 32 or can be loaded in needle 32 after needle 32 has been advanced through distal anchor delivery device 30. Distal anchor 12 is advanced through needle 32 such that it emerges out of the distal end of needle 32. In alternate embodiments, the distal anchor can be held in place by a pusher or connector while the needle is retracted, thus exposing the distal anchor.

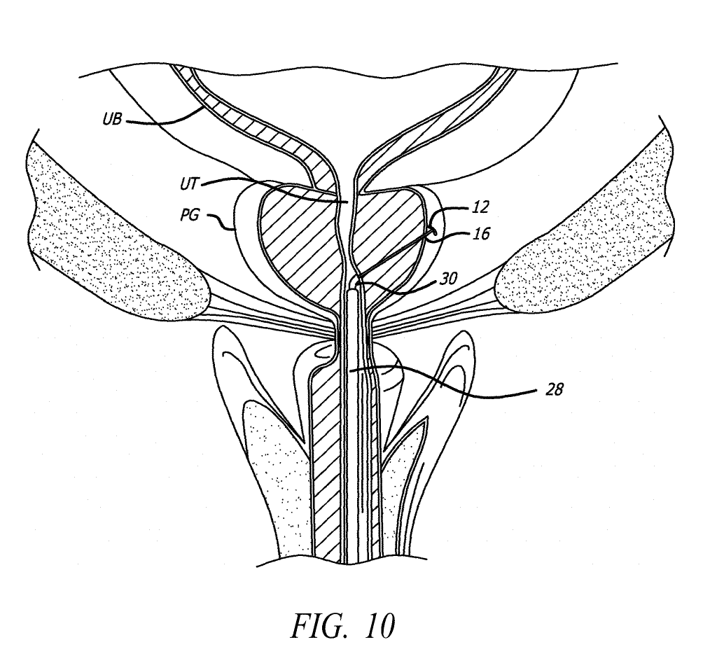

In the step shown in FIG. 10, needle 32 is removed from distal anchor delivery device 30 by pulling needle 32 in the proximal direction.

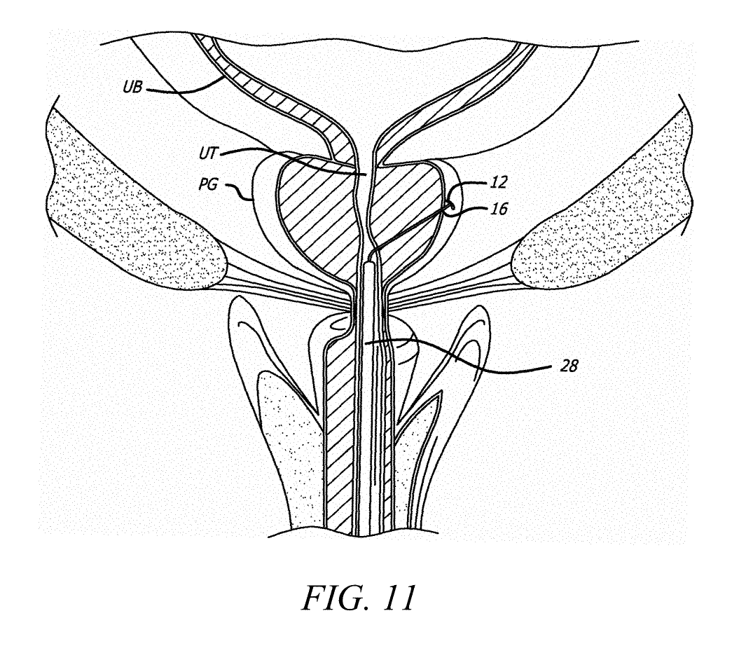

In the step shown in FIG. 11, distal anchor delivery device 30 is removed from sheath 28 by pulling distal anchor delivery device 30 in the proximal direction. Also, connector 16 is pulled to orient distal anchor 12 perpendicularly to connector 16.

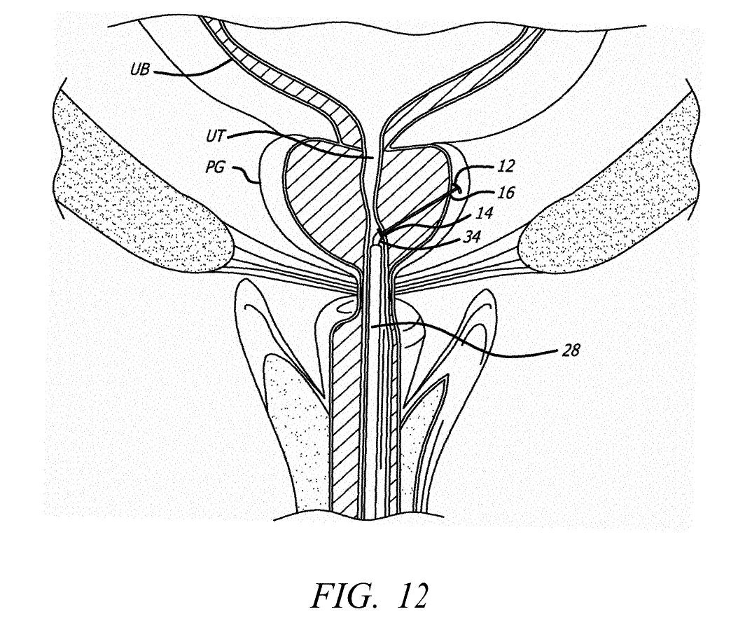

In the step shown in FIG. 12, connector 16 is passed through proximal anchor 14 located on a proximal anchor delivery device 34. Proximal anchor delivery device 34 is advanced through sheath 28 such that the distal end of proximal anchor delivery device 34 emerges out of the distal end of sheath 28. A desired tension is introduced in connector 16 such that distal anchor 12 is pulled by connector 16 with a desired force. Alternatively, the proximal anchor can be visualized through an endoscope or under fluoroscopy and advanced along the connector until the desired retraction of the tissue is achieved. In other embodiments, the proximal anchor is a v-shaped or clothespin-shaped piece that is forced, in some cases at high speed, onto the connector to fixedly engage the connector.

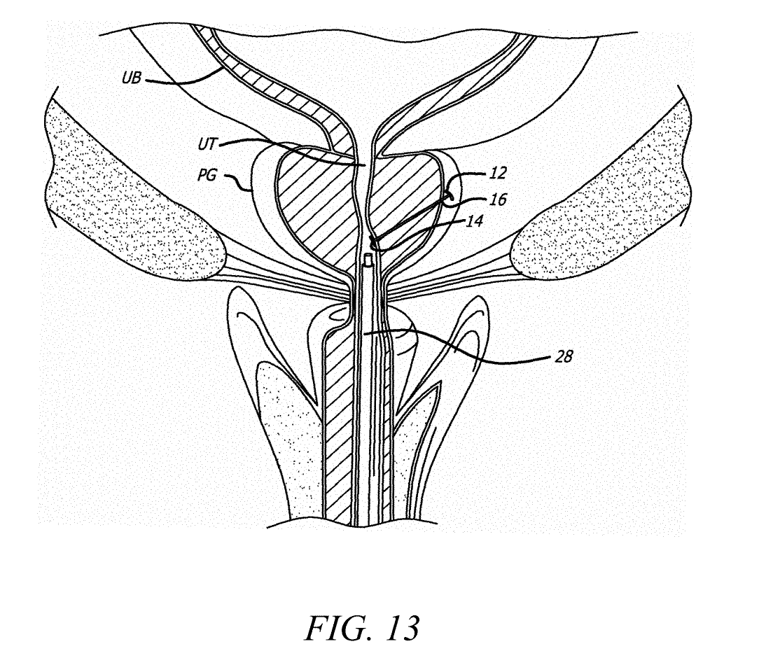

In the step shown in FIG. 13, connector 16 is attached to proximal anchor 14. Proximal anchor 14 is also released from proximal anchor delivery device 34, thus deploying proximal anchor 14 in the anatomy. Proximal anchor delivery device 34 and sheath 28 are removed form the anatomy. Retainer 10 comprising distal anchor 12, proximal anchor 14 and connector 16 is used to retract, lift, support, reposition or compress a region of prostate gland PG located between distal anchor 12 and proximal anchor 14. This method may be used to retract, lift, support, reposition or compress multiple regions or lobes of the prostate gland PG. In the method shown in FIGS. 7 through 13, distal anchor 12 is deployed on the outer surface of the capsule of prostate gland CP. Thus, distal anchor 12 acts as a capsular anchor. Alternatively, distal anchor 12 may be deployed inside the tissue of prostate gland PG or beyond the prostate as outlined previously. Similarly, in the method shown in FIGS. 7 through 13, proximal anchor 14 is deployed on the inner wall of urethra UT and acts as a urethral anchor. Alternatively, proximal anchor 14 may be deployed inside the tissue of prostate gland PG. Moreover, the positive pressure bulb 41 can be attached to the sheath assembly throughout the procedure and manipulated to displace tissue or other matter present at the interventional site.

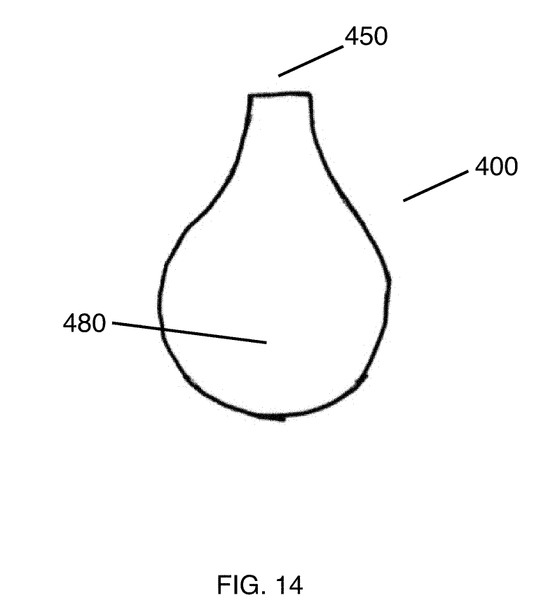

FIG. 14 illustrates one embodiment of a positive pressure reservoir. In this embodiment, a positive pressure bulb 400 has a coupling port 450 that is designed to attach to a sheath assembly. The coupling port 450 can attach via a luer lock, threads, a press-fit, a suction fit, or other attachment means. The junction between coupling port 450 and the sheath assembly is preferably fluid tight. Embodiments of the positive pressure bulb 400 can include adapters such that coupling port 450 is capable of coupling to a variety of sheath assembly designs and forming the aforementioned fluid tight connection. The various adapters present the different means of attachments, including, but not limited to, a luer lock, threads, a press-fit, a suction fit, or other attachment means.

Referring still to FIG. 14, the positive pressure bulb 400 includes handling region 480, which is shaped to fit comfortably within a users hand and allow for single-hand operation of the positive pressure bulb 400. The handling region 480 can have a textured surface to facilitate ease of gripping. The handling region 480 can be more of less compliant than other regions of the positive pressure bulb 400 to facilitate the single-handed application of pressure. The desired compliance of handling region 480 can be achieved by choice of materials, such as a more flexible or a less flexible material. The flexibility of the material can be chosen by varying the thickness of a given material, making a composite of materials with varying flexibility, or other similar means. The designs of positive pressure bulb 400 are not limited to a single bulb. Multiple bulb embodiments may be used such that a user can easily vary the pressure delivered to the sheath assembly by choosing to manipulate a given bulb, and this manipulation can be accomplished with a single hand. Still further, the positive pressure bulb 400 can optionally include inlet ports such that other sources of fluid can be used in conjunction with the fluid found in the sheath assembly. In this way, the positive pressure bulb 400 can be used to facilitate delivery of a particular fluid content to the interventional site.

Embodiments described herein provide several advantages, including, but not limited to, providing a path for visualization of an interventional site. In some embodiments, the changes in fluid pressure created by the positive pressure reservoir can improve the operative conditions at the interventional site or enhance the efficacy of the intervention. For example, the changes in fluid pressure created by the positive pressure reservoir can facilitate administration of contrast solution or medications. Contrast solution can improve direct and indirect visualization. Medications can include vasomanipulative drugs to affect the local conditions of the tissue and can include other drugs to accomplish other localized effects. Changes in fluid pressure created by the positive pressure reservoir can create transient fluid or pressure waves for therapeutic purposes.

Further, the devices and methods disclosed herein can be used to treat a variety of pathologies in a variety of lumens or organs comprising a cavity or a wall. Examples of such lumens or organs include, but are not limited to urethra, bowel, stomach, esophagus, trachea, bronchii, bronchial passageways, veins (e.g. for treating varicose veins or valvular insufficiency), arteries, lymphatic vessels, ureters, bladder, cardiac atria or ventricles, uterus, fallopian tubes, etc.

In certain embodiments, the changes in fluid pressure created by the positive pressure reservoir can be used to affect other parts of the therapeutic system. For example, changes in fluid pressure created by the positive pressure reservoir can be used to flush parts of a sheath or introducer or other similar medical devices where the parts of the device being flushed are not proximate the interventional site. The positive pressure apparatus can be configured to mate with supply lines or drainage lines. That is, the changes in fluid pressure created by the positive pressure reservoir can be used to positively affect the fluid supply path to an interventional site or the drainage path away from an interventional site. In these embodiments, the changes in fluid pressure created by the positive pressure reservoir improves the operative conditions even though the direct effect of the pressure change is experienced in a part of the medical apparatus that is not directly proximate the interventional site.

Finally, it is to be appreciated that the disclosure has been described hereabove with reference to certain examples or embodiments of the disclosure but that various additions, deletions, alterations and modifications may be made to those examples and embodiments without departing from the intended spirit and scope of the disclosure. For example, any element or attribute of one embodiment or example may be incorporated into or used with another embodiment or example, unless to do so would render the embodiment or example unpatentable or unsuitable for its intended use. Also, for example, where the steps of a method are described or listed in a particular order, the order of such steps may be changed unless to do so would render the method unpatentable or unsuitable for its intended use. All reasonable additions, deletions, modifications and alterations are to be considered equivalents of the described examples and embodiments and are to be included within the scope of the following claims.

Thus, it will be apparent from the foregoing that, while particular forms of the disclosure have been illustrated and described, various modifications can be made without parting from the spirit and scope of the disclosure.

We claim:

1. A system for an interventional procedure, comprising:
an interventional device;
a sheath assembly including an elongated tubular portion extending from a main body, the elongate tube portion sized and shaped to receive the interventional device, the main body including a plurality of ports; and
a positive pressure reservoir, wherein manipulation of the positive pressure reservoir transmits pressure through the elongate tube.

2. The system of claim 1 wherein the positive pressure reservoir comprises a bulb.

3. The system of claim 1 wherein the positive pressure reservoir comprises a syringe.

4. The system of claim 1 wherein the positive pressure reservoir is configured to mate with at least one of the plurality of ports.

5. The system of claim 1 wherein the positive pressure reservoir is configured to mate with a line connected to at least one of the plurality of ports.

6. The system of claim 1, wherein the elongate tube portion includes a terminal end and manipulation of the positive pressure reservoir creates fluid pressure and fluid motion.

7. The system of claim 1, wherein the elongate tube portion is sized and shaped to extend within a body orifice, and extends to an interventional site.

8. The system of claim 1, further comprising a scope, wherein manipulation of the positive pressure reservoir provides a path for visualization by the scope.

9. The system of claim 8, wherein manipulation of the positive pressure reservoir results in cleaning the scope.

10. The system of claim 1, wherein manipulation of the positive pressure reservoir affects the operative conditions at an interventional site.

11. The system of claim 1, wherein manipulation of the positive pressure reservoir delivers a therapeutic fluid to an interventional site.

12. The system of claim 1, further comprising a fluid source connected to one of the plurality of ports.

13. The system of claim 1 wherein the anchor assembly comprises a first anchor, a connector, and a second anchor.

14. A method for treatment at an interventional site, comprising:
providing a sheath assembly, the sheath assembly including an elongate tube portion having a terminal end and a main body including a plurality of ports;
providing an interventional device sized and shaped to be received within the elongate tube portion of the sheath assembly;
providing a scope for visualizing the interventional site;
inserting a sheath assembly into a patient's body and configuring the terminal end of the elongate tube portion at an interventional site;
placing the scope and interventional device in position at an interventional site;
attaching a positive pressure reservoir to one of the plurality of ports of the sheath assembly; and
manipulating the positive pressure bulb to generate fluid pressure and flow through the elongate tube portion.

15. The method of claim 14, further comprising attaching a fluid source to one of the plurality of ports of the sheath assembly.

16. The method of claim 14, wherein manipulating the positive pressure reservoir creates a path of visualization for the scope.
